(19) United States
(12) Patent Application Publication
(10) Pub. No.: US 2011/0218632 A1
(43) Pub. Date: Sep. 8, 2011

(54) SURGICAL IMPLANTS

(75) Inventors: Christopher Reah, Taunton (GB); Alan McLeod, Somerset (GB)

(73) Assignee: NUVASIVE, INC., San Diego, CA (US)

(21) Appl. No.: 13/107,557

(22) Filed: May 13, 2011

Related U.S. Application Data
(63) Continuation of application No. 11/597,161, filed on Aug. 27, 2007.

(30) Foreign Application Priority Data
May 20, 2004 (GB) GB 0411249.6
Sep. 30, 2004 (GB) GB 0421635.4

(51) Int. Cl. A61F 2/44

(52) U.S. Cl. 623/17.16

(57) ABSTRACT

Systems and methods for replacement of an intervertebral disc are provided. The spinal implant comprises an elastomeric material that has been treated with an additive to decrease the ability of emissions to pass through the elastomeric material and to increase at least one of wear resistance, stiffness, and Shore hardness of the elastomeric material. The additive may be a barium-containing compound, one or more metals, or one or more x-ray opaque materials. The additive may be included in certain localities of the implant. A series of implants may be provided with different sizes and different structural properties that mimic the stiffness and shock absorbing properties of the natural intervertebral disc to be replaced.

This invention concerns improvements in and relating to surgical implants, particularly, but not exclusively in relation to surgical implants for the replacement of intervertebral discs in the spine.

Increasingly there is a desire to address problems with intervertebral discs by replacing all or part of the disc with a prosthetic disc, rather than fusing the adjacent vertebrae. A wide variety of designs of disc prostheses exist, including: articulated metal plates with a spring or like between them; metal end plates with a polyethylene spacer; and a spacer of elastomeric or visco-elastic material in a retaining fabric, U.S. Pat. No. 6,093,205. In the later two cases, the same material for the spacer is used in all examples of that type of prosthesis, irrespective of the prosthesis's size.

The present invention has amongst its aims to provide an improved partial or total spinal disc replacement of a size tailored to be appropriate to a given patient. The present invention has amongst its aims to provide a spinal disc replacement whose properties more accurately reflect the actual natural disc to be replaced.

According to a first aspect of the present invention we provide a series of disc prostheses, the series including at least a first prosthesis and a second prosthesis, the first prosthesis differing in size and differing in one or more materials from which it is formed compared with the second prosthesis, the first and second prostheses being of the same type.

The series may include at least 3 prostheses, preferably at least 4 prosthesis, more preferably at least 5 prostheses and potentially 10 or more. The series may include a number of prostheses which is at least half the number of intervertebral disc spaces in the human spine or in a part thereof, such as the cervical part or lumbar part. The series may include a number of prostheses equal to the number of intervertebral disc spaces in the human spine or in a part thereof, such as the cervical part or lumbar part.

The series may be divided into two or more sets of prostheses, one of which includes the aforesaid first and second prostheses. Preferably two or more of the sets, and ideally all of the sets, include a first prosthesis and a second prosthesis, the first prosthesis of a set differing in size and differing in one or more structural properties compared with the second prosthesis of a set. Two or more sets may include prostheses which are of the same size as each other, but which differ between the sets in terms of one or more of their structural properties. Two or more sets may include sizes of prostheses which are matched in size by prostheses in the second set.

Different sets of prostheses may be intended for different uses. The different uses may be to address different medical conditions and/or medical procedures and/or different patients. The different patients may differ in terms of their build and/or intervertebral disc spacing and/or natural intervertebral disc properties.

All the disc prostheses of a series and/or each set of prostheses may be of the same type. The type of disc prosthesis may be defined by its general structure.

One type of disc prosthesis may be provided as a pair of, preferably rigid or semi-rigid, end plates with a core between them. The core may be bonded to the end plates. The core may be unbonded to the end plates. The core may be partially bonded to the end plates. The end plates may be metallic.

Another type of disc prosthesis may be provided by a core which is encapsulated, preferably within a fabric. The core may be constrained by the encapsulation. The core may be retained by the encapsulation. The core may be partially encapsulated.

The prostheses may differ in size from one another so as to approximate to, and ideally match, the size of the natural intervertebral disc they are intended to replace. The prostheses may differ in one or more structural properties relative to one another so as to approximate to, and ideally match, one or more of the structural properties of the natural intervertebral disc they are intended to replace. Preferably the prostheses approximate to, and ideally match a plurality, and ideally all, of the structural properties of the natural intervertebral disc they are intended to replace.

The series may include a prosthesis intended to be a specific match for each intervertebral disc space in a human spine. The series may include a prosthesis intended to be a specific match for each intervertebral disc space in a part of the human spine, for instance the cervical part and/or the lumbar part.

The first prosthesis and second prosthesis may differ in size in terms of one or more or all of their cross-sectional area (ideally considered in plan in the position of use in the spine), width (ideally considered across the disc space in use), depth (ideally considered from posterior to anterior in use) and height (ideally concerned in the direction of separation of adjacent vertebrae in use). The difference may apply to the whole of a size determining parameter or only a part thereof. For instance, the maximum and/or minimum and/or location specific values may be being considered when providing prostheses which differ in size from one another.

The first prosthesis and second prosthesis may differ in material so as to control one or more structural properties of the prosthesis. The one or more structural properties may include the compressional and/or axial stiffness. The one or more structural properties may include the flexion extension stiffness. The one or more structural properties may relate to the extent of deformation under compressive load and/or the bending stiffness and/or the shock absorbing capability and/or the rotational stiffness.

The differences in size and/or material and/or one or more structural properties may be provided by the prosthesis as a whole and/or by a part thereof The part thereof may particularly be the core or a component thereof. The differences in size and/or material and/or one or more structural properties may be provided by the end plates, particularly in the end plate and core type prosthesis, potentially with the core constant in size and/or material and/or structural properties between different prostheses. The differences in size and/or material and/or one or more structural properties may be provided by the core or a component thereof, particularly in the encapsulated core type prosthesis, potentially with the encapsulation being constant in material and/or structural properties between different prostheses.

The material of the end plates may differ between the first prosthesis and the second prosthesis. The material of the encapsulation may differ between the first prosthesis and the second prosthesis. Preferably the material of the core may differ between the first and the second prosthesis.

The material, particularly the material of the core, may differ due to the material used to form it and/or the grade of material used to form it and/or due to one or more additives and/or the level thereof. The material, particularly the material of the core may differ due to the treatment of the material before and/or during and/or after production. In particular, the material may differ due to the curing conditions to which it is subjected. The core may be caused to differ by varying a component thereof, for instance a filler.

The same type may be a type in which the first and second prostheses comprise a pair of, preferably rigid or semi-rigid, end plates with a core between them. The same type may be a type in which the first and second prostheses comprise a core which is encapsulated, preferably within a fabric. The same type may be a type in which the first and second prostheses comprise common structure features and/or types of components.

The first aspect of the invention may include any of the features, options or possibilities set out elsewhere in this application.

According to a second aspect of the present invention we provide a method for producing disc prostheses, the method including forming disc prostheses of a first kind and forming disc prostheses of a second kind, the first prosthesis kind differing in size and differing in one or more materials from which it is formed compared with the second prosthesis kind, the first and second prostheses being of the same type.

The method may include choosing the material of the first prosthesis and/or second prosthesis to provide one or more structural properties. The material may be chosen to provide one or more structural properties matched to a particular disc in a human spine and/or of a particular person. The material may be chosen to provide a particular compressional/axial stiffness and/or flexion extension stiffness. The material may be chosen to give a compressional/axial stiffness and/or flexion extension stiffness which is the same as a particular disc in a human spine and/or of a particular person. The material may be chosen to give a compressional/axial stiffness and/or flexion extension stiffness which is less than that of a particular disc in a human spine and/or of a particular person. The material may be chosen to give a compressional/axial stiffness and/or flexion extension stiffness which is greater than that of a particular disc in a human spine and/or of a particular person.

The second aspect of the invention may include any of the features, options or possibilities set out elsewhere in this application.

According to a third aspect of the present invention we provide a surgical method for provide a disc prosthesis, the method including removing at least a part of the natural disc in a spine and inserting a disc prosthesis in the spine, the disc prosthesis being selected from a series of disc prostheses, the series including at least a first prosthesis and a second prosthesis, the first prosthesis differing in size and differing in one or more materials from which it is formed compared with the second prosthesis, the first and second prostheses being of the same type.

The third aspect of the invention may include any of the features, options or possibilities set out elsewhere in this application.

According to a fourth aspect of the present invention we provide a series of disc prostheses, the series including at least a first prosthesis and a second prosthesis, the first prosthesis differing in size and differing in one or more materials from which it is formed compared with the second prosthesis, the first and second prostheses being of the same type, the type providing a pair of, preferably rigid or semi-rigid, end plates with a core between them.

The core may be bonded to the end plates. The core may be unbonded to the end plates. The core may be partially bonded to the end plates. The end plates may be metallic.

According to a fifth aspect of the present invention we provide a series of disc prostheses, the series including at least a first prosthesis and a second prosthesis, the first prosthesis differing in size and differing in one or more materials from which it is formed compared with the second prosthesis, the first and second prostheses being of the same type, the type being a core which is encapsulated, preferably within a fabric.

According to a sixth aspect of the present invention we provide a series of disc prostheses, the series including at least a first prosthesis and a second prosthesis, the first prosthesis differing in size and differing in one or more materials from which it is formed compared with the second prosthesis, the first and second prostheses being of the same type, the type being any type other than a type providing a pair of, preferably rigid or semi-rigid, end plates with a core between them.

According to a seventh aspect of the present invention we provide a series of disc prostheses, the series including at least a first prosthesis and a second prosthesis, the first prosthesis differing in size and differing in one or more materials from which it is formed compared with the second prosthesis, the first and second prostheses being of the same type, the type being any type other than a type providing a core which is encapsulated, preferably within a fabric.

The fourth and/or fifth and/or sixth and/or seventh aspects of the invention may include any of the features, options or possibilities set out elsewhere in this application.

According to an eighth aspect of the invention we provide a medical device, the device including a first part and a second part, the first part having a first set of properties, the second part differing from the first part with respect to one or more of those properties.

The medical device may be an implant. The medical device may be an implant for the spine. The medical device may be one or more of a prosthetic disc, a partial disc prosthesis, a fusion device, a nucleus pulposis replacement, an annulus replacement, a nucleus pulposis repair, an annulus repair. The medical device may be a joint replacement. The medical device may be tubing. The medical device may be an implant or device used in minimally invasive surgery. The medical device may be or be part of an instrument.

The top surface and/or bottom surface and/or sides of a medical device or component thereof may be provided by the first part(s). The top and bottom layers of the medical device or component thereof may be provided by the first part(s). An intermediate layer of the medical device or component thereof may be provided by the first part(s). One or two side portions of the medical device or component thereof may be provided by the first part(s). The majority of the medical device or component thereof may be provided by the first part(s). A plug and/or insert of the medical device or component thereof may be provided by the first part(s). One or more flexible members of the medical device or component thereof may be provided by the first part(s). The flexible members may be elongate and/or longitudinally aligned with one another and/or surrounded by an at least partial enclosure.

The top surface and/or bottom surface and/or sides of a medical device or component thereof may be provided by the second part(s). The top and bottom layers of the medical device or component thereof may be provided by the second part(s). An intermediate layer of the medical device or component thereof may be provided by the second part(s). One or two side portions of the medical device or component thereof may be provided by the second part(s). The majority of the medical device or component thereof may be provided by the second part(s). A plug and/or insert of the medical device or component thereof may be provided by the second part(s). One or more flexible members of the medical device or component thereof may be provided by the second part(s). The flexible members may be elongate and/or longitudinally aligned with one another and/or surrounded by an at least partial enclosure.

The first part may include an element embedded therein. The element may be wire and/or band and/or strip. Preferably the element provides the difference in one or more properties.

The second part may include an element embedded therein. The element may be wire and/or band and/or strip. Preferably the element provides the difference in one or more properties.

The first part may be provided at one or more localities in the medical device. One or more of the localities may be an edge of the medical device and/or an edge of a component of the medical device. One or more of the localities may be a face of the medical device and/or a component of the medical device. The edge and/or face may be at the posterior of the medical device and/or a component thereof, particularly in the context of a medical device for the spine. The edge and/or face may be at the anterior of the medical device and/or a component thereof, particularly in the context of a medical device for the spine.

The second part may be provided at one or more localities in the medical device. One or more of the localities may be an edge of the medical device and/or an edge of a component of the medical device. One or more of the localities may be a face of the medical device and/or a component of the medical device. The edge and/or face may be at the posterior of the medical device and/or a component thereof, particularly in the context of a medical device for the spine. The edge and/or face may be at the anterior of the medical device and/or a component thereof, particularly in the context of a medical device for the spine.

The medical device may be formed of one or more first parts and one or more second parts. The first part(s) and/or second part(s) may be provided in layers.

The one or more first parts and one or more second parts are preferably joined together. Preferably an integral unit is formed of the first and second parts.

The second part may differ from the first part in terms of a lower ability of an emission to pass through the second part compared with the first part. The second part may differ from the first part in terms of a higher ability of an emission to pass through the second part compared with the first. The emissions may be generated externally of the body containing the device. The emission may be radiowaves. The emission may be x-rays. The emission may be gamma rays. The emissions may be generated internally in the body containing the device. The emissions may arise as a result of magnetic resonance imaging. The emissions may arise as a result of the application of a magnetic field and/or radiowaves to the body.

The first part may be substantially opaque to one or more emission types. The first part may be opaque to one or more emission types. The first part may be opaque to x-rays. The second part may be substantially opaque to one or more emission types. The second part may be opaque to one or more emission types. The second part may be opaque to x-rays.

The first part may be different to the second part due to one or more additional materials being present. The additional materials may be present in the first and/or second part. The additional material may be present in one part and absent from the other part. The additional material may be present in one part at one level and present in the other part at a different level. The additional material may be one or more barium containing compounds and/or one or more metals and/or one or more x-ray opaque materials. The additional material may be or include barium sulphate. The additional material may be between 1 wt % and 50 wt % of the part. The additional material may more preferably be between 8 wt % and 20 wt % of the part.

The second part may differ from the first part in terms of a lower wear resistance and/or stiffness and/or Shore hardness compared with the first part. The second part may differ from the first part in terms of a higher wear resistance and/or stiffness and/or Shore hardness compared with the first part.

The first part may be different to the second part in terms of its wear resistance and/or stiffness and/or Shore hardness due to one or more additional materials being present. The additional materials may be present in the first and/or second part. The additional material may be present in one part and absent from the other part. The additional material may be present in one part at one level and present in the other part at a different level. The additional material may be one or more barium containing compounds and/or one or more metals and/or one or more x-ray opaque materials. The additional material may be or include barium sulphate. The additional material may be between 1 wt % and 50 wt % of the part. The additional material may more preferably be between 8 wt % and 20 wt % of the part.

Preferably the second part differs from the first part in terms of a lower ability of an emission to pass through the second part compared with the first part and one or more of a higher wear resistance and/or greater stiffness and/or greater Shore hardness. Preferably the second part differs from the first part in terms of a higher ability of an emission to pass through the second part compared with the first and one or more of a lower wear resistance and/or greater stiffness and/or greater Shore hardness. The second part may differ from the first part in terms of a lower ability of an emission to pass through the second part compared with the first part and one or more of a lower wear resistance and/or lesser stiffness and/or lesser Shore hardness. The second part may differ from the first part in terms of a higher ability of an emission to pass through the second part compared with the first and one or more of a higher wear resistance and/or lesser stiffness and/or lesser Shore hardness.

Preferably the first part may be different to the second part, in respect of its ability for an emission to pass through and one or more of wear resistance and/or stiffness and/or Shore hardness, due to the same one or more additional materials being present. The same additional material may be present in the first and/or second part. The same additional material may be present in one part and absent from the other part. The same additional material may be present in one part at one level and present in the other part at a different level. The same additional material may be one or more barium containing compounds and/or one or more metals and/or one or more x-ray opaque materials. The same additional material may be or include barium sulphate. The same additional material may be between 1 wt % and 50 wt % of the part. The same additional material may more preferably be between 8 wt % and 20 wt % of the part.

The eighth aspect of the invention may include any of the features, options or possibilities set out elsewhere in this application and in particular in the ninth and/or tenth and/or eleventh aspects of the invention.

According to a ninth aspect of the invention we provide the use of an additive decrease the ability of emissions to pass through a material and to increase the wear resistance and/or stiffness and/or Shore hardness of a material.

Preferably the ability of emissions to pass through the material refers to the ability of x-rays to pass through the material. Preferably the wear resistance of the material is increased. Preferably the additive is x-ray opaque. Preferably the additive is a barium compound. More preferably the additive includes barium sulphate. Ideally the barium compound is provided at between 8 wt % and 20 wt % of the material.

The ninth aspect of the invention may include any of the features, options or possibilities set out elsewhere in this application and in particular in the eighth and/or tenth and/or eleventh aspects of the invention.

According to a tenth aspect of the invention we provide a method of producing a medical device, the method including providing a device including a first part and a second part, the first part being provided with a first set of properties, the second part differing from the first part with respect to one or more of those properties.

The first and second parts are preferably joined together. The first and second parts may be joined together as part of the production process, for instance in a mould. The first and second parts may be separate and then joined together. The first and second parts may be formed at the same time, for instance by introducing them at the same time, for instance into the same mould. The first and second parts may be formed by calanderising. One or more first parts may be cut from a large piece. One of more second parts may be cut from a large piece. The pieces may be sheets. The parts may be put together as a series of layers. The first and second parts may alternate in terms of the layers. The first and/or second parts may be uncured. Preferably the first and/or second parts are cured after assembly. The first and second parts may be joined to one another by bonding and/or gluing.

The first part may differ from the second part due to one of the parts having an additional material. The additional material may be provided in the first part and/or the second part prior to the first and second parts being joined. The additional material may be provided in the first part and/or the second part after the first and second parts are joined. The additional material may be provided by injection.

The tenth aspect of the invention may include any of the features, options or possibilities set out elsewhere in this application and in particular in the eighth and/or ninth and/or eleventh aspects of the invention.

According to an eleventh aspect of the invention we provide a method in which a medical device is provided in a body, the device including a first part and a second part, the first part having a first set of properties, the second part differing from the first part with respect to one or more of those properties and one or more of the properties are investigated.

The medical device may be provided in the body by surgery. The body may be human. Preferably the method determines the position of the medical device in the body. The determination of position may be with reference to one or more first parts of the medical device and one or more features of the body. The determination of position may be with reference to one or more second parts of the medical device and one or more features of the body. The method may include adjusting the position of the medical device in the body. The investigation may be by x-ray. The investigation may be by way of magnetic resonance imaging.

The eleventh aspect of the invention may include any of the features, options or possibilities set out elsewhere in this application and in particular in the eighth and/or ninth and/or tenth aspects of the invention.

Various embodiments of the invention will now be described, by way of example only, and with reference to the accompanying drawings in which:

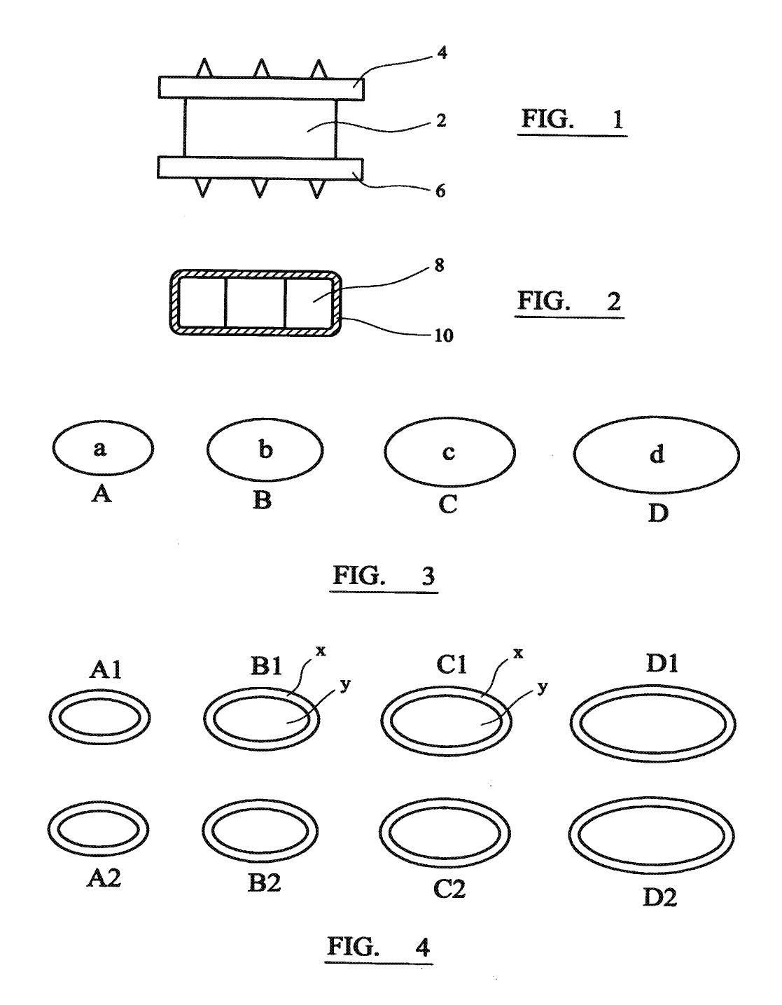

FIG. 1 is an illustration of a first type of prosthesis which can be provided according to the present invention;

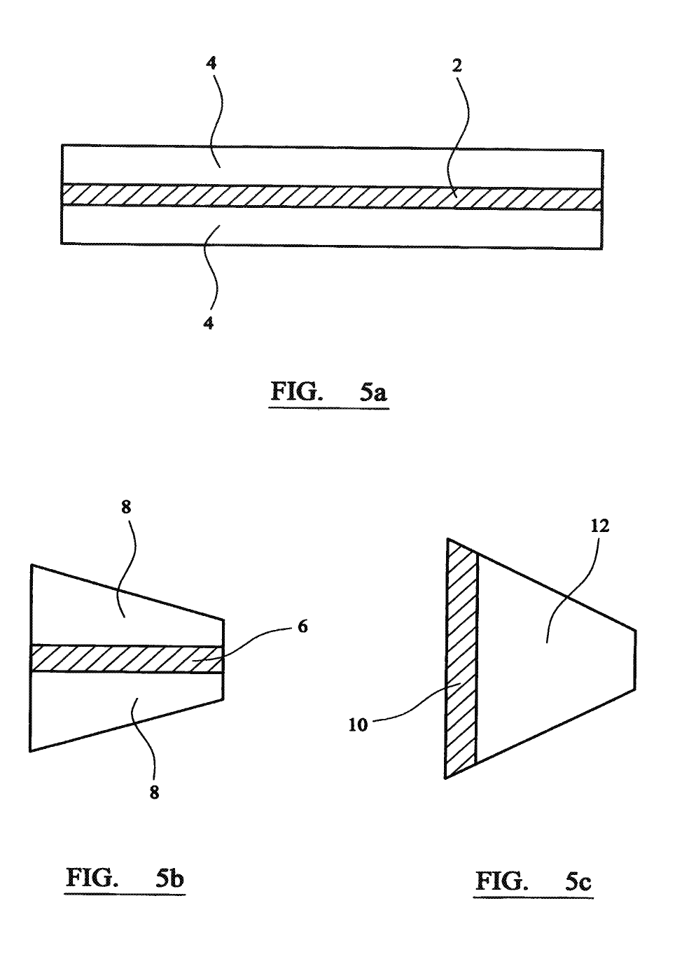

FIG. 2 is an illustration of a second type of prosthesis which can be provided according to the present invention;

FIG. 3 is an illustration of a set of cores for use in prostheses and provided according to one embodiment of the present invention;

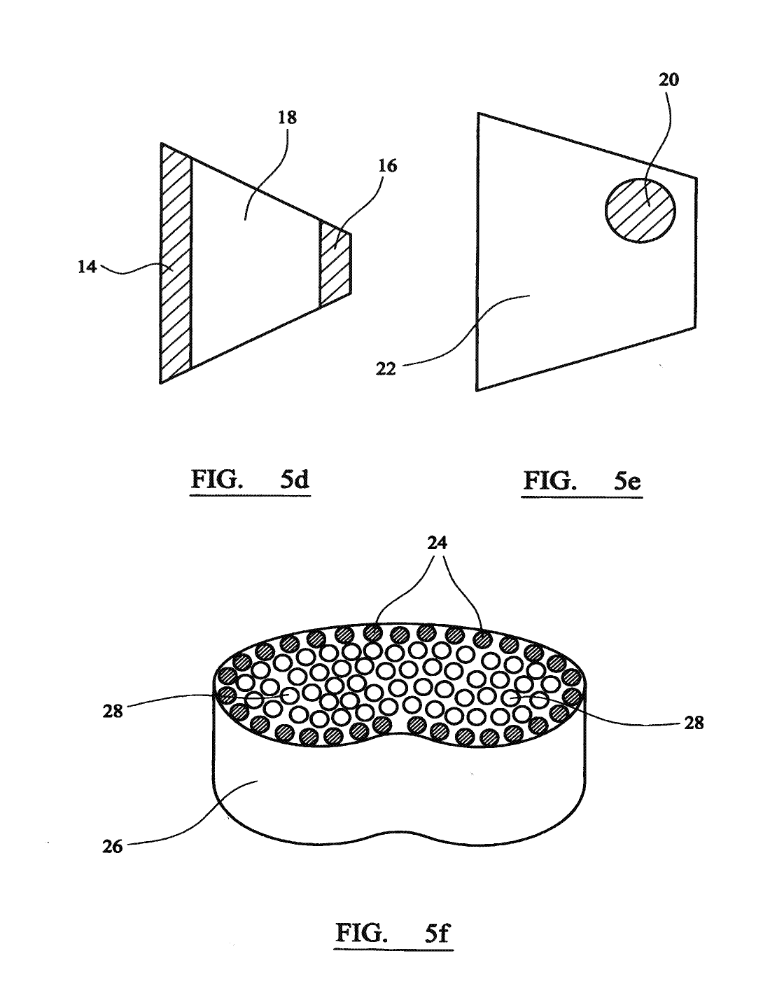

FIG. 4 is an illustration of a set of cores for use in prostheses and provided according to a second embodiment of the present invention;

FIGS. 5a to 5f are illustrations of embodiments of the invention which make use of localised variations in properties.

Many medical implants come in a variety of sizes to fit the patient. The choice of size is usually determined by the best fit to the patient. In the case of most joints this is not an issue as the joints either rotate freely (such as hip and knee joints) or do not require specific stiffness qualities to operate (such as small finger or ankle joints).

Developments in intervertebral disc replacements include new designs of disc based upon using the deformation of an elastomer to allow motion of the intervertebral joint. Elastomer designs include:

Bonded elastomers, see FIG. 1, where the elastomer 2 is bonded between rigid or very stiff end-plates 4, 6 that are usually metallic in nature.

Encapsulated elastomers, see FIG. 2, where the elastomer 8 is surrounded by another material 10, such as a fabric.

For each design type, a single elastomer material is chosen, for instance a polyethylene, and exactly the same material is used in all the elastomers of that design type. Such designs seek to provide some deformation under compressive loads, some bending stiffness and some shock absorbing capability. Ideally they seek to have a compressive stiffness, rotational stiffness and/or shock absorbing capability that mimics the natural disc.

As intervertebral discs come in a variety of sizes, depending upon their position in the spine, it is desirable to provide a variety of different sizes of artificial intervertertebral discs to most closely match the size of the patient's natural disc. Presently, this is done by scaling up or down the design, but using the same materials in each case.

Significant problems arise from simply scaling a standard design of artificial disc in either cross section and/or height as this is likely to result in each size of artificial intervertebral disc having different compressive and bending stiffness' and also different shock absorbing capabilities.

For example, if the disc has a constant thickness then doubling the cross sectional area will approximately double the compressive stiffness. If the disc has a constant cross sectional area then doubling the thickness of the elastomer will approximately half the compressive stiffness. Whilst the effects are not exact multiples, they are very close.

This variation in properties is not a true reflection of the variation in properties for natural intervertebral discs with size. As a result, the replacements are not totally accurate mimics of the natural discs.

Attempting to use a single sized disc in all patients is not an effective solution. Such a disc would have to be of the smallest size encountered in any vertebrae of any patient and so would be of relatively small size. This has the disadvantage of not passing the load through the entire vertebral body and could result in the disc subsiding in the patient.

This problem could be combated to an extent by using a series of variable sized end plates, together with a common spacer design. The end plates may or may not have different angulations to allow different lordosis angles. Thus a common spacer is provided which can be connected to or detached from end plates so as to allow it to be combined with different end plate sizes. The material of one spacer of the common design may differ in its material in a selected way from another spacer of the same design.

Another embodiment of the approach taken in the present invention is illustrated in FIG. 3. A series of different disc sizes are provided, with the surgeon potentially selecting the appropriate size so as to mimic the size of natural disc being replaced. As well as mimicking size, however, the invention also provides that the elastomer used in each different size has a different Shore hardness. This allows the intended stiffness and/or shock absorbing properties to be provided in each disc size and so provides for full mimicking of the natural disc in each size. Rather than taking the prior art approach of scaling the size and living with the consequences in terms of the structural properties which result, the present invention also provides for careful selection of the material so as to provide the desired properties too.

In the series shown in FIG. 3, there are four different sizes, A, B, C and D, each larger than the last in cross-sectional area (shown) and depth (not shown). Of course, the variation between discs may be in respect of one or more size factors. Four different Shore hardness elastomers are used, a for A, b for B, c for C and d for D. The elastomer is basically the same general type of elastomer in each case.

In the embodiment of the invention illustrated in FIG. 4, the concept is expanded further. Here the series includes a first set of four different sizes A1, B1, C1 and D1 and a second set of four sizes, each matching to one size in the first set, the sizes being A2, B2, C2 and D2. The first set is intended for a first type of situation requiring disc replacement and the second set is intended for a second type of situation requiring disc replacement. The different situation may be a different medical condition and/or different type of patient and/or, as in this embodiment, a different build of patient. Thus for the first set, intended perhaps for patients of larger build, the Shore hardness is greater for the same size of disc than the Shore harness of the second set. Disc A1 is harder than disc A2, disc B1 is harder than disc B2 and so on to reflect the greater loads to be accommodated. Within the sets the Shore hardness varies between the different sizes too. Thus D1 is softer than C1, is softer in turn than B1, is softer in turn than A1. The second set is intended for a lighter build of patient.

A further difference in the FIG. 4 embodiment is the manner in which the elastomer is formed. In this case, the elastomer provides an outer X which contains a filler Y. Embodiments in which the filler Y is distributed, preferably as evenly as possible, throughout the elastomer may be provided. Different filler types and/or concentrations of filler are used in this embodiment to vary the Shore hardness.

As well enabling the correct size and properties for a medical implant so as to suit a wide variety of people, the present invention also achieves other benefits through the use of carefully selected additives and structures for implants.

After, and even potentially during surgery, it is helpful to be able to establish the position of an implant accurately. The applicant has added material to the elastomer which forms an implant, during its production, to make it visible to x-ray inspection. One way to do so is mix in barium compounds, such as barium sulphate. A loading of 13% by weight represents an example of this approach. The barium sulphate is thus distributed throughout the elastomer and does make the implant visible on x-ray images.

Such an approach has been improved upon, however, as this technique of rendering all of the implant opaque can give rise to implants which are opaque to x-rays to too high an extent. This can impair the information on the position of the implant which can be discerned from the x-ray and/or obscure other features which would otherwise be visible in the image.

The improved approach provides the x-ray opaque material in only certain localities within the elastomer. As a result, the extent of opaqueness can be controlled and yet full positional information on the implant be provided.

A variety of methods for providing localised x-ray opaqueness are possible.

The x-ray opaque material can provided in one part of an implant, with the other part x-ray opaque material free. Within the mould, the two parts can be joined together to form the overall implant and yet maintain the localised distribution of the x-ray opaque material. An implant in one continuous piece is thus provided. The x-ray opaque material can be introduced as a powder and/or as a filler.

When forming the implant in a mould, it is also possible to provide two separate injection nozzles with one introducing the material to form one part of the implant and the other introducing material to form another part of the implant. One of the nozzles provides the material with the x-ray opaque material in it. The materials may only differ from one another in terms of the x-ray opaque material or may have other differences.

Calanderising is another possible production technique. Different parts of the eventual implant are cut out of blocks or sheets of different materials. The materials differ from one another at least in terms of one possessing the x-ray opaque material. The different parts are put together as a series of layers of uncured elastomer and then joined together, for instance in a mould. Thus opaque and non-opaque layers could be alternated, for instance.

Other ways of joining the different parts of the eventual implant together also exist. Chemical reactions, the use of bonding agents, other forms of bonding and gluing are all possible ways of fixing one or more parts formed of a material lacking the x-ray opaque material to one or more other parts of the material provided with the x-ray opaque material.

The x-ray opaque material could be injected into a formed elastomer, for instance during the setting and/or curing stage to give localised distribution in that way.

Unexpectedly, the applicant has also established that the provision of x-ray opaque material within the elastomer can render the parts that contain the x-ray opaque material harder wearing than those parts without. Thus the x-ray opaque material can be used for this purpose alone and/or to achieve the desired visibility under x-rays.

Whilst barium containing compounds and the like have been mentioned above, other forms of x-ray opaque material for localised use, exist. These include the use of wires or metal beads in the elastomer. Potentially such forms of the invention also offer structural/wear resistance improvements too. In some cases, it may be desirable to form one or more parts of the implant/item weaker than others. Such parts include those where the implant is designed to fold during assembly and/or during insertion into the patient.

The provision of implants and the like which have limited and/or localised radioopacity and/or radiolucency and/or limited and/or localised strength and/or limited or localised stiffness offers many advantages and possibilities.

It should also be noted, that the material added to localities within the implant or other item could be used to modify the appearance of the implant/item to other imaging approaches. Alterations to render localities on the implant highly visible to MRI and other imaging approaches are possible.

Whilst the concept of localised x-ray opaque material is described above mainly in the context of implants for the spine, the concept is broadly applicable. It could be used in a wide variety of other temporary or permanent implants (small joint replacements, tubing and the like) and/or minimal invasive surgery situations where visibility in x-rays is desired. The concept is applicable to a very wide range of elastomers, such as silicones.

By way of example, some possible structures with localised x-ray opaque material are shown in FIG. 5a through FIG. 5f.

In FIG. 5a, a part with x-ray opaque material 2 is sandwiched between two parts 4 which are free of x-ray opaque material. The parts 2, 4 are joined to one another to form an integral implant.

In FIG. 5b, a part 6 which includes x-ray opaque material is shown between parts 8 which are x-ray opaque material free. The non-parallel faces of the parts 8 reflect overall configurations encountered in spinal implants.

In FIG. 5c, the x-ray opaque material is restricted to one face 10 of the implant, with the other part 12 being x-ray opaque material free. Such a set up is particularly useful for clearly indicating the posterior and/or anterior edge of the implant when inserted.

In FIG. 5d, both the posterior edge 14 and anterior edge 16 are shown with x-ray opaque material, whilst the middle of the implant, part 18, is x-ray opaque material free. This approach allows good judgement of the position of the implant, when viewed from the side of the patient. The side view is the most readily used view in an x-ray. This approach also means that the amount of the image rendered opaque by the x-ray opaque material is minimised; the image will show two lines, with transparent material between.

In FIG. 5e, a small plug 20 of x-ray opaque material is provided in a larger part 22 which is x-ray opaque material free. Many possibilities are present for the localised x-ray opaque material.

Finally in FIG. 5f, some of the many parts 24 aligned with one another within a sleeve 26 are provided with x-ray opaque material and other parts 28 are not.

What is claimed is:

1. A method of performing disc replacement surgery comprising:
removing at least a part of a natural intervertebral disc;
selecting a surgical implant for replacement of at least a part of the natural intervertebral disc that comprises an elastomeric material that has been treated with an additive to decrease the ability of emissions to pass through the elastomeric material and to increase at least one of wear resistance, stiffness, and Shore hardness of the elastomeric material, wherein the additive is varied to provide a surgical implant having different structural properties that mimic the natural intervertebral disc to be replaced; and
implanting the surgical implant having the selected structural properties in a body.

2. The method of claim 1 further comprising:
determining a position of the surgical implant after implantation.

3. The method of claim 2 further comprising:
adjusting the position of the surgical implant after implantation.

4. The method of claim 1, wherein the ability of emissions to pass through the elastomeric material refers to the ability of x-rays to pass through the material.

5. The method of claim 1, wherein the additive is a barium compound.

6. The method of claim 5, wherein the barium compound is barium sulphate.

7. The method of claim 1, wherein the additive is from 1 wt % to 50 wt % of the elastomeric material.

8. The method of claim 1, wherein the additive is from 8 wt % to 20 wt % of the elastomeric material.

9. The method of claim 1, wherein the structural properties are at least one of stiffness and shock absorbing properties.

10. The method of claim 9, wherein the stiffness is at least one of compressional stiffness, axial stiffness, flexion extension stiffness, bending stiffness, and rotational stiffness.

11. The method of claim 1, wherein the additive is distributed throughout the elastomeric material.

12. The method of claim 1, wherein the additive is included in only certain localities within the elastomeric material.

13. The method of claim 1, wherein the additive is included in only certain localities of the elastomeric material by including the additive in a part sandwiched between two parts which are free from the additive.

14. The method of claim 1, wherein the additive is included in only certain localities of the elastomeric material by including the additive in a part that is restricted to a single face of the surgical implant with another part that is free from the additive.

15. The method of claim 1, wherein the additive is included in only certain localities of the elastomeric material by including the additive on both a posterior edge and an anterior edge of the surgical implant with a middle part of the surgical implant that is free from the additive.

16. The method of claim 1, wherein the additive is included in only certain localities of the elastomeric material by including the additive in a small plug that is provided in a larger part that is free from the additive.

17. The method of claim 1, wherein the additive is included in only certain localities of the elastomeric material by including the additive in parts that align with one another within a sleeve with other parts that are free from the additive.
